(19) United States
(12) Patent Application Publication
(10) Pub. No.: US 2008/0145317 A1
(43) Pub. Date: Jun. 19, 2008

(54) EFFERVESCENT FORMULATIONS OF FLORFENICOL FOR ADDITION IN DRINKING WATER SYSTEMS

(75) Inventors: SERENA TONGIANI, Cranford, NJ (US); Keith A. Freehauf, Stockton, NJ (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(73) Assignee: Schering Corporation

(21) Appl. No.: 11/957,668

(22) Filed: Dec. 17, 2007

Related U.S. Application Data
(60) Provisional application No. 60/870,705, filed on Dec. 19, 2006.

(51) Int. Cl. A61K 9/12; A61P 31/04

(52) U.S. Cl. 424/43

(57) ABSTRACT

The invention relates to an effervescent formulation containing florfenicol or an antibiotic of related structure. When introduced into water, the effervescent formulation is dissolved with minimal or no agitation and provides a solution or a homogeneous dispersion of florfenicol. The formulations are typically in the form of free flowing powders, granules or tablets. The effervescent formulations are useful for the treatment of bacterial infections and allow for the rapid dispersion and dissolution of florfenicol in the drinking water systems of an animal in need of such treatment.

This Application claims the benefit of U.S. Provisional Application No. 60/870,705, filed Dec. 19, 2006

### FIELD OF THE INVENTION

The present invention relates to an effervescent pharmaceutical composition of florfenicol (FFC), and antibiotics of related structure in the form of blend powder, granules or tablets. The composition is providing a rapidly solubilizing additive for addition to drinking water systems for the treatment of bacterial infections in animals.

### BACKGROUND OF INVENTION

Florfenicol is a broad spectrum antibiotic, structurally related to thiomphenicol and chloramphenicol, which has been developed as a veterinary treatment for use in animals, particularly cattle, swine, poultry, and fish.

Florfenicol is indicated for the control of mortality due to E. coli airsacculitis in broiler chickens, and for treatment and control of swine respiratory diseases associated with Actinobacillus pleuropneumoniae, P. multocida, Mycoplasma, Salmonella cholera suis and Streptococcus suis Type II. Currently veterinary products containing florfenicol are available in the form of injectable (for treatment of cattle respiratory diseases) powder or granules (addition to feed in aquaculture) and as concentrate nonaqueous organic solution (for addition in swine and poultry drinking water systems), The effervescent product designed for addition to drinking water systems has significant appeal to the industry because of its ease of administration. One currently marketed product is Nuflor® Concentrate Solution (Schering-Plough). Nuflor® Concentrate Solution uses a high content of organic solvent to provide an immediate solubilization of florfenicol in water and can be used not only in the bulk water source, at the efficacious concentration of 400 mg/gal (˜0.1 mg/mL), but also with an automated proportioner mixing tank system (mixing ratio of 1 oz. to 1 gallon) at a concentration of ˜13.5 mg/mL. There are, however, some drawbacks associated with an organic concentrate solution that would be eliminated with the use of a soluble powder formulation. For example, the bottles in which the concentrate solution is dispensed create disposal and storage issues, especially for large facilities that need to treat more than one drinking water line at the same time. Furthermore, a limited expiration date has been observed for this product. A soluble powder formulation could overcome issues associated with the concentrate organic solution. For example, an improved powder composition would ideally be expected to be a stable dosage form with an extended shelf life. It would also be ideally dispensed in smaller packages and thus pose less problems with disposal. Furthermore, improved products should be formulated without including any sugar based fillers such as lactose or sucrose, so as to prevent any possible problem of bacteria or mold growth in the drinking water lines.

The delivery of florfenicol as soluble powder in the drinking water system is not an easy task. One of the challenges is its relatively low water-solubility (1.23 mg/mL). Another challenge associated with developing a soluble powder containing florfenicol is the drug's limited wettability in water. Upon addition to water, florfenicol floats on the surface and does not disperse evenly throughout the volume of water. Over the years, various techniques have been suggested to overcome these issues. Various pro-drug formulations and other solubilization techniques such as the use of surfactants and encapsulation, have been proposed (U.S. Pat. No. 7,122,198). Recently, efforts have been directed to provide novel formulations containing florfenicol. When added to water, such formulations would rapidly disperse and dissolve the florfenicol with minimal or no agitation. It would also offer advantages over the existing florfenicol-based forms of treatment. In addition, such products could have a high level of user acceptability. In order to be useful and gain acceptance in the field, the above mentioned issues relating to the difficulties associated with providing florfenicol in aqueous solutions would have to be solved, and the present invention addresses these needs.

### SUMMARY OF THE INVENTION

The effervescent formulations of the invention are typically in the form of free flowing powders, granules or tablets. In accordance therewith, one aspect of the invention provides effervescent formulations that contain:
a) from about 10 to about 65 wt % florfenicol;
b) from about 20 to about 80 wt % of an alkaline component; and
c) from about 0 to about 60 wt % of an acid component.

The formulations are capable of effervescence in the presence of moisture and can rapidly disperse and dissolve the florfenicol with minimal or no agitation in water.

In some particular aspects of the invention, the ratio of the alkaline component to the acid component is greater than 1:1. One particular alkaline component used in the effervescent formulations is sodium hydrogen carbonate. Some particular acid components include citric acid, tartaric acid and mixtures thereof.

In other aspects of the invention there are provided methods of preparing the effervescent formulations, methods of treatment using the same and kits containing the same.

### DETAILED DESCRIPTION OF THE INVENTION

The invention provides effervescent formulations comprising florfenicol for use in animal drinking water system. In some particular aspects of the invention, the effervescent formulations are stable powders, granules or compressed tablets that can be added directly to the drinking water system to reach the antibiotic therapeutic dose with a fast solubility rate profile.

For purposes of the present invention, “effervescent couple” shall be understood to include the acid and alkaline components that generate gas upon reaction in the presence of moisture or water.

For purposes of the present invention, “animal” shall be understood to include, but not be limited to, swine, cattle, poultry or any domesticated animal intentionally reared in an agricultural setting to make produce such as food or fiber or for its labor.

One of the key components of the effervescent formulations of the invention is the drug florfenicol. Florfenicol can be prepared as an effervescent formulation as free base or in its salt form and also in any of its derivatives form such as phosphate derivatives and any florfenicol pro-drugs. Florfenicol is not hygroscopic, so its incorporation in an effervescent formulation does not cause instability due to water absorption. Florfenicol is also known as [R-(R*, S*)]-2,2-Dichloro-N-[1-(fluoromethyl)-2-hydroxy-2-[4-[(methylsulfonyl)phenyl]-ethyl]acetamide. Processes for the manufacture of this preferred antibiotic, and intermediates useful in such processes, are described in U.S. Pat. Nos. 4,311,857; 4,582,918; 4,973,750; 4,876,352; 5,227,494; 4,743,700; 5,567,844; 5,105,009; 5,382,673; 5,352,832; and 5,663,361. Another preferred antibiotic is thiamphenicol. Pharmaceutically-acceptable salts of the foregoing are also contemplated for addition to the effervescent formulations described herein.

In some aspects of the invention, the amount of florfenicol included in the effervescent formulation may range from about 10% to about 65% by weight. In some particular aspects, the amount of florfenicol is from about 20 to about 50% by weight of the formulation, while in other particular aspects, the amount is from about 40 to about 50% by weight. In one particular aspect the amount is about 20%.

The effervescent formulations preferably contain an acid and alkaline mixture that generates carbon dioxide upon contact with water. Alternatively any effervescent materials that energetically evolve gasses upon contact with water can be incorporated. The alkaline component of the couple is particularly present in excess of the stoichiometric equivalent of the acid component, i.e. the ratio of alkaline component to acid component is greater than 1:1. In some particular aspects of the invention, the ratio of alkaline component to acid component is from about 1.1:1 to about 1.5:1. In another particular aspect, the ratio between acidic and basic component is stoichiometrically calculated to be about 1:1.44.

With the foregoing ratios in mind, the amount of the alkaline component included in the effervescent formulations is from about 20 to about 80% by wt of the formulation, and particularly from about 20 to about 50% by wt, and particularly from about 30 to about 45% by wt (˜30-45% in examples). In another particular aspect, the amount of an alkaline component included in the effervescent formulation is about 41% by wt of the formulation. The amount of the acid component in the effervescent formulations described herein can range from about 0 to about 60% by wt, particularly from about 20 to about 40% by wt, and particularly from about 30 to about 40% by wt (˜30-40% in examples). In another aspect, the amount of acid component in the effervescent formulation described herein is about 36% by wt of the formulation.

The alkaline component is particularly a carbonate or bicarbonate salt such as potassium bicarbonate, calcium carbonate, sodium hydrogen carbonate, or mixtures thereof. In one particular aspect of the invention, the alkaline component is sodium hydrogen carbonate. As will be appreciated by those of ordinary skill, alternatives may be used. A non-limiting list of alternatives includes salts suitable for use in illustrative embodiments include, but are not limited to, the alkali metal salts. sodium carbonate, magnesium carbonate, ammonium carbonate, potassium carbonate, sodium bicarbonate, and calcium bicarbonate, mixtures thereof and the like such as alkali metal carbonates or bicarbonates may all be employed, as well as those materials well known in the art of effervescent materials.

The acid component of the effervescent formulation can be selected from among a wide variety of pharmaceutically acceptable acids. A non-limiting list includes, but is not limited to, citric acid, tartaric acid, malic acid, fumaric acid, adipic acid, oxalic acid, sulfamic acid, mixtures thereof and the like. In particular, acids included are citric acid, tartaric acid, fumaric acid malic acid, mixtures thereof and the like. As will be appreciated by those of ordinary skill, the foregoing is merely illustrative. Combinations of the foregoing as well as mixtures with other acids not specifically mentioned herein are also contemplated.

Conventional excipients, such as colorants, fillers, diluents, surfactants, sweeteners, flavorants, preservatives, antioxidants, stabilizers, as well as other ancillary pharmaceutically acceptable ingredients and the like and mixtures thereof may be added to the formulations. For example, the formulations can also contain additional common excipients such us binders, lubricants, diluents, surfactants, solvents and mixtures thereof. One particular diluent is lactose anhydrous. Other diluents that are suitable include without limitation microcrystalline cellulose, sorbitol, starch and calcium phosphate. The amount of diluent can range from about 0% by wt. to about 40% by wt. One particular lubricant is magnesium stearate but other suitable lubricants can include, without limitation, calcium phosphate and/or calcium phosphate dibasic. The amount of lubricant can range from about 0% by wt. to about 5% by wt. One particular surfactant is Tween80, but other suitable surfactants can include, without limitation, sodium lauryl sulfate. The amount of surfactant can range from about 0% by wt. to about 10% by wt. One particular binder is polyvinylpyrrolidone (PVP) 30. The amount of binder can range from about 2 to about 20% by wt in aqueous or alcoholic solution. A non-limiting list of suitable alternatives may include polyvinylpyrrolidone 90, starch, methylcellulose, sodium carboxymethylcellulose, polyacrilamide and polyvinyl alcohols.

Other optional inert ingredients may be added to the present composition, as desired. Such ingredients include preservatives, antioxidants, stabilizers, colorants, sweeteners and flavorants. Exemplary preservatives include methyl p-hydroxybenzoate (methylparaben) and propyl p-hydroxybenzoate (propylparaben). Exemplary antioxidants include butylated hydroxyanisole and sodium monothioglycerol. Particular stabilizers for use in the present invention include, for example, BHT or citric acid. One particular stabilizer to prevent degradation of any of the active ingredients in the formulations of the present invention is BHT in a concentration between about 0.01% (w/w) and about 0.05% (w/w). Other suitable stabilizers include, for example fumaric acid, malic acid, and tartaric acid. When a suitable acid is used as a preservative, it can be added in addition to or as part of the acid component, according with the stoichiometric ratio between the acid and basic components in the effervescent formulation.

Exemplary sweeteners are mannitol, lactose, sucrose and dextrose.

Some particular aspects of the invention include an effervescent couple (the combination of alkaline and acid components, which together form the effervescence in aqueous solutions), which is preferably composed of citric acid and sodium hydrogen carbonate or tartaric acid and sodium hydrogen carbonate. However, other solid acid/carbonate couples may be substituted. For example, sodium glycine carbonate or malic acid and sodium carbonate or potassium bicarbonate or any combination of these acid and alkaline components can be used.

Exemplary solvents include, but are not limited to, ethanol and water. Ethanol is preferred to prevent the effervescent reaction during a wet granulation process.

In still further aspects of the invention, the effervescent formulations may contain a second pharmaceutically active composition that does not interfere or otherwise hamper the effectiveness of the florfenicol. It will be appreciated that other active ingredients may be combined with the formulations of the present invention. Such ingredients may include, for example, anti-inflammatory agents such as corticosteroids, NSAIDS, such as flunixin, COX-inhibitors and other analgesics, antiparasitic compounds such as, for example, an avermectin compound such as ivermectin, doramectin, milbemycin, selamectin, emamectin, eprinomectin, and moxidectin, and/or optionally a flukicide. It may also be preferred to employ a second antibiotic in the formulation. Preferred antibiotics may include tetracyclines. Particularly preferred is chlorotetracycline and oxytetracycline. Other preferred additional antibiotics include beta-lactams, such as penicillins, cephalosporins, e.g., penicillin, amoxicillin, or a combination of amoxicillin with clavulanic acid or other beta lactamase inhibitors, ceftiofur, cefquinome, etc. Additional preferred antibiotics include fluoroquinolones, such as, for example, enrofloxacin, danofloxacin, difloxacin, orbifloxacin and marbofloxacin, and macrolide antibiotics such as tilmicosin, tulathromycin, erythromycin, azithromycin and pharmaceutically-acceptable salts there of and the like. Alternatively, one could include insect growth regulators in combination with the formulations of the present invention.

In order to prepare the composition of the present invention the effervescent mixture, i.e. the alkaline component and acid component are prepared in a stoichiometric ratio in which the alkaline component is present in a stoichiometric excess to that of the acid component. The florfenicol and any optional active is incorporated therein and dry blended. If a blended powder is the final product, flavorants, sweeteners, preservatives and antioxidants, if added, are incorporated at this point. Alternatively, if a wet granulation is performed, a solution containing the selected binder and solvent is prepared and added to the mixture. The preparation is mixed until suitable granulation is achieved. Flavorants, sweeteners, preservatives and antioxidants, if added, are incorporated in the binder/solvent solution. The granules are then dried, milled and screened to the desired size. The formulations described herein can also be prepared via direct compression.

In another aspect of the invention there are provided methods of treating or preventing bacterial infections and florfenicol-susceptible conditions. The methods include introducing a sufficient amount of an effervescent formulation as described herein into water, and administering the resultant solution to an animal in need thereof, as part of the liquid to be ingested by the animal, e.g., the formulation may be added into its drinking water system to administer the treatment and therapeutic dose to an animal in need of such treatment.

The amount administered is a therapeutically- or prophylactically-effective amount of the florfenicol solution resulting from the introduction of the effervescent formulation and water. In most aspects of this embodiment, the amount of effervescent formulation added to water is an amount that is sufficient to bring the concentration of florfenicol in the drinking water to from about 0.05 mg/mL to about 25 mg/mL. Particularly, the concentration of florfenicol in the drinking water will be from about 0.01 mg/mL to about 0.2 mg/mL. Particularly, the concentration will be about 0.1 in the bulk drinking water, and a concentration of about 13.5±0.1 mg/mL when the aqueous solutions are used in a typical proportioner mixing ratio of 1:128 gallons. Depending upon the condition being treated and the type, size, weight, etc. of the animal being treated, it is contemplated that suitable periods of treatment will range from about 1 to about 5 days or longer if needed using the novel formulations in drinking water at the concentrations mentioned above. As will be appreciated by those of ordinary skill, the animals will drink the treated water ad libitum. It is nonetheless contemplated that sufficient amounts of the florfenicol will be administered to the animals in need thereof when it is available for drinking for the periods mentioned above.

The effervescent compositions of the present invention may, if desired, be presented in a pack or dispenser device, such as an FDA approved kit, which may contain one or more unit dosage forms containing the effervescent formulation in the form of compressed tablets, granules or powder containing the active ingredient. The pack may, for example, comprise metal or plastic foil, such as a blister pack. The pack may also consist of a soluble biodegradable pouch ready to use, sealed in a metal plastic foil. The pack or dispenser device may be accompanied by instructions for administration. The pack or dispenser may also be accompanied by a notice associated with the container in a form prescribed by a governmental agency regulating the manufacture, use or sale of pharmaceuticals, which notice is reflective of approval by the agency of the form of the compositions or of human or veterinary administration. Such notice, for example, may be of the labeling approved by the U.S. Food and Drug Administration for prescription drugs or of an approved product insert. Compositions comprising a compound of the invention formulated in a compatible pharmaceutical carrier may also be prepared, placed in an appropriate container, and labeled for treatment of an indicated condition. Thus, the kit can be used in connection with the treating or preventing of a bacterial infection in an animal in need thereof and include a sufficient amount of the effervescent formulation described herein and instructions for introducing the effervescent formulation into drinking water to be given to the animal in need thereof.

### EXAMPLES

The following examples are provided to illustrate certain embodiments of this invention and are not intended, nor are they to be construed, to limit its scope in any manner whatsoever.

In Examples 1-5, effervescent formulations were made according to the formulae provided in each table and made according to the following steps.

Citric acid and Tartaric acid are mixed in a suitable container and sodium hydrogen carbonate is added. Florfenicol is dry blended with the effervescent mixture until homogeneity is achieved. A solution of PVP is prepared in ethanol and slowly added to the dry components. The mixture is mixed until a suitable granulation point is achieved. Additional components, if present, such as surfactants, flavorants or antioxidants are added to the PVP solution. The granules are then dried, milled and screened.

### Example 1

| INGREDIENT | % (w/w) |
|---|---|
| Florfenicol | 20 |
| Citric Acid | 12 |
| Tartaric Acid | 24 |
| Sodium Hydrogen carbonate | 41 |
| PVP30 | 3 |

### Example 2

| INGREDIENT | % (w/w) |
|---|---|
| Florfenicol | 20 |
| Citric Acid | 11.25 |
| Tartaric Acid | 22.5 |
| Sodium Hydrogen carbonate | 38.2 |
| PVP30 | 3 |
| Tween80 | 5 |

### Example 3

| INGREDIENT | % (w/w) |
|---|---|
| Florfenicol | 20 |
| Citric Acid | 11.6 |
| Tartaric Acid | 23.2 |
| Sodium Hydrogen carbonate | 39.4 |
| PVP30 | 3 |
| Sodium Lauryl Sulfate | 2.5 |

### Example 4

| INGREDIENT | % (w/w) |
|---|---|
| Florfenicol | 20 |
| Citric Acid | 31 |
| Sodium Hydrogen carbonate | 43.7 |
| PVP30 | 5 |

### Example 5

| INGREDIENT | % (w/w) |
|---|---|
| Florfenicol | 20 |
| Citric Acid | 31 |
| Sodium Hydrogen carbonate | 43.4 |
| PVP30 | 3 |
| Sodium Lauryl Sulfate | 2,5 |

To determine the effectiveness of the effervescent formulations, a dissolution test was performed using a USP apparatus 2 with paddle agitation at 50 rpm. The dissolution medium was Milli-Q water maintained at a temperature of 25° C. The effervescent formulations were added directly to the water in the correct amount to achieve a final concentration of 0.1 mg/mL of florfenicol. Aliquots of the resulting solution were withdrawn and analyzed using either UV-VIS spectrophotometry or HPLC, the latter to exclude excipient contribution or degradation of florfenicol. For HPLC analysis, an organic/aqueous mobile phase was used for the separation on a C18, reverse phase column. Detection was performed by UV absorption spectrometry. The percent dissolved was calculated versus an external reference standard prepared at the nominal concentration of the analyte.

Pure florfenicol was analyzed as a comparator to assess the effectiveness of the effervescent formulation. Referring now to the tables below, it can be seen that pure florfenicol was only 25% dissolved in 15 minutes and 43% dissolved within 60 minutes. By comparison, florfenicol formulated as an effervescent mixture as per Example 1 was 97% dissolved in 5 minutes and 100% dissolved within 60 minutes. Therefore, the effervescent formulation increased the florfenicol rate of dissolution and allowed a complete dissolution within 5 minutes of the addition to water.

TABLE 1
Solubility of Pure Florfenicol in water

| Time | % Drug in Solution |
|---|---|
| 0 | 0 |
| 15 | 25 |
| 30 | 35 |
| 45 | 36 |
| 60 | 43 |

TABLE 2
Solubility Rate Profile of Florfenicol Formulation of Example 1

| Time | % Drug in Solution* |
|---|---|
| 0 | 0 |
| 1 | 91 |
| 3 | 96 |
| 5 | 97 |
| 10 | 99 |
| 15 | 100 |
| 30 | 100 |
| 45 | 100 |
| 60 | 100 |

*All data in Table II are normalized to 100%

Although certain presently particular embodiments of the invention have been described herein, it will be apparent to those skilled in the art to which the invention pertains that variations and modifications of the described embodiments may be made without departing from the spirit and scope of the invention.

Accordingly, it is intended that the invention be limited only to the extent required by the appended claims and the applicable rules of law.

What is claimed is:

1. An effervescent formulation comprising:
a) from about 10 to about 65 wt % florfenicol;
b) from about 20 to about 80 wt % of an alkaline component; and
c) from about 0 to about 60 wt % of an acid component, said formulation capable of effervescence in the presence of moisture.

2. The effervescent formulation of claim 1, wherein the florfenicol is from about 20 to about 50 wt % of the formulation.

3. The effervescent formulation of claim 2, wherein the florfenicol is from about 40 to about 50 wt % of the formulation

4. The effervescent formulation of claim 1, wherein the ratio of alkaline component to acid component is greater than 1:1.

5. The effervescent formulation of claim 4, wherein the ratio of alkaline component to acid component is from about 1.1:1 to about 1.5:1.

6. The effervescent formulation of claim 1, wherein the alkaline component is from about 20 to about 50 wt % of the formulation.

7. The effervescent formulation of claim 6, wherein the alkaline component is from about 30 to about 45 wt % of the formulation

8. The effervescent formulation of claim 1, wherein the acid component is from about 20 to about 40 wt % of the formulation.

9. The effervescent formulation of claim 8, wherein the acid component is from about 30 to about 40 wt % of the formulation.

10. The effervescent formulation of claim 1, wherein the alkaline component is a carbonate or bicarbonate salt.

11. The effervescent formulation of claim 10, wherein the alkaline component is selected from the group consisting of potassium bicarbonate, calcium carbonate, sodium hydrogen carbonate and mixtures thereof.

12. The effervescent formulation of claim 11, wherein the alkaline component is sodium hydrogen carbonate.

13. The effervescent formulation of claim 1, wherein the acid component is selected from the group consisting of citric acid, tartaric acid, malic acid, fumaric acid, adipic acid, oxalic acid, sulfamic acid, and mixtures thereof.

14. The effervescent formulation of claim 13, wherein the acid component is selected from the group consisting of citric acid, tartaric acid and mixtures thereof.

15. The effervescent formulation of claim 1, further comprising a member of the group consisting of preservatives, antioxidants, stabilizers, colorants, sweeteners, flavorants, binders, diluents, lubricants, surfactants, solvents, fillers and mixtures thereof.

16. The effervescent formulation of claim 15, wherein the binder is PVP 30.

17. The effervescent formulation of claim 15, wherein the binder is present in an amount ranging from about 2 to about 20% by wt.

18. The effervescent formulation of claim 1, further comprising a second pharmaceutically active composition.

19. The effervescent formulation of claim 18, wherein the second pharmaceutically active composition is flunixin.

20. The effervescent formulation of claim 18, wherein the second pharmaceutically active composition is COX-2 inhibitor.

21. The effervescent formulation of claim 18, wherein the second pharmaceutically active composition is an avermectin.

22. A method of treating or preventing a bacterial infection, comprising
introducing a sufficient amount of the effervescent formulation of claim 1 into water, and administering to an animal in need thereof a therapeutically- or prophylactically-effective amount of the product resulting from the introduction of said effervescent formulation and water.

23. The method of claim 17, wherein the concentration of florfenicol administered to said animal is from about 0.01 to about 0.2 mg/ml.

24. A kit for treating or preventing a bacterial infection in an animal in need thereof, comprising a sufficient amount of the effervescent formulation of claim 1 and instructions for introducing the effervescent formulation into drinking water given to the animal in need thereof.

25. An effervescent formulation comprising:
a) about 20 wt % florfenicol;
b) about 41 wt % of an alkaline component; and
c) about 36 wt % of an acid component, said formulation capable of effervescence in the presence of moisture.
